(19) United States
(12) Patent Application Publication
(10) Pub. No.: US 2009/0202649 A1
(43) Pub. Date: Aug. 13, 2009

(54) FENOFIBRATE FORMULATIONS

(76) Inventors: Subhash Gore, Sholapur (IN); Anand Sankaranarayanan, Chennai (IN); Balaji Sathurappan, Tirunelveli (IN); Raviraj Sukumar Pillai, Hyderabad (IN); Pradip Kumar Ghosh, Midnapur West (IN); S.H. Seyed Mohamed Buhary, Tirunelveli (IN); Sainath Kalisetty, Cuddapah (IN); Pratit Premchand Agrawal, Gondia (IN); Manikandan Ramalingam, Kanchaepuram (IN); Venugopal Kumaran, Chennai (IN); Ajay Kumar Reddy Thupalli, Kadapa (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/366,230

(22) Filed: Feb. 5, 2009

Related U.S. Application Data
(60) Provisional application No. 61/046,562, filed on Apr. 21, 2008.

(30) Foreign Application Priority Data
Feb. 6, 2008 (IN) 317/CHE/2008

(51) Int. Cl. A61K 9/14; A61K 31/216; A61P 9/00

(52) U.S. Cl. 424/490; 514/545

(57) ABSTRACT

The present invention relates to pharmaceutical formulations comprising fenofibrate. The invention also relates to stable and bioavailable pharmaceutical formulations comprising fenofibrate. Further the invention also relates to processes for preparing the compositions and/or formulations of fenofibrate and their methods of use, treatment and administration.

### INTRODUCTION

The present invention relates to pharmaceutical formulations comprising fenofibrate. The invention also relates to pharmaceutical formulations comprising compositions of fenofibrate. Further the invention also relates to processes for preparing the compositions and/or formulations of fenofibrate and their methods of use, treatment and administration.

The present invention as discussed in various embodiments relates to a stable and bioavailable formulations comprising fenofibrate.

Fenofibrate is a lipid regulating agent. A chemical name for fenofibrate is 2-[4-(4-chlorobenzoyl) phenoxy]-2-methyl-propanoic acid, 1-methylethyl ester. It has a molecular formula C₂₀H₂₁O₄Cl, molecular weight of 360.83, and has structural Formula I.

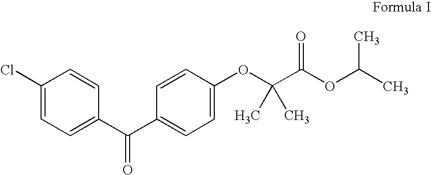

Commercially available products containing fenofibrate include TriCor® tablets, sold by Abbott Laboratories and containing either 48 mg or 145 mg of fenofibrate. TriCor tablets are indicated for the treatment of hypertriglyceridemia and hypercholesterolemia.

Since fenofibrate has a very low aqueous solubility, a large challenge for the formulator is development of an oral dosage form with enhanced dissolution properties.

Fenofibrate and its preparation are described in U.S. Pat. No. 4,058,552. Fenofibrate has been prepared in several different formulations. U.S. Pat. Nos. 4,800,079 and 4,895,726 disclose a co-micronized formulation of fenofibrate and a solid surfactant.

U.S. Pat. Nos. 6,652,881, 6,589,552, 6,596,317 and 7,041,319, disclose compositions and/or formulations of micronized fenofibrate.

U.S. Pat. Nos. 5,145,684, 6375,986, 7,276,249, 7,320,802, disclose nanoparticulate compositions and/or formulations of fenofibrate with a surface stabilizer.

U.S. Pat. Nos. 6,277,405 and 7,037,529, 6,076,670, 6,596,317, disclose fenofibrate formulations with improved dissolution profiles.

U.S. Pat. No. 6,828,334 discloses an inclusion complex of fenofibrate with cyclodextrins. U.S. Pat. No. 6,027,747 discloses solid dispersions of fenofibrate.

U.S. Pat. No. 6,368,622 discloses a process for preparing a formulation, having the steps of forming a melt granulate of a fibrate with a surfactant.

U.S. Patent Application Publication Nos. 2004/0087656, 2007/0298115, 2008/0138424, and 2008/0241070 describe fenofibrate having particle sizes less than 2000 nm with an improved bioavailability.

International Application Publication No. WO 2004/028506 discloses an oral pharmaceutical formulation of fenofibrate.

Even though various approaches have been attempted to make fenofibrate formulations, there remains a need for improved formulations in which the fenofibrate exhibits better dissolution properties. The invention relates to stable and bioavailable formulations comprising fenofibrate.

### SUMMARY

The present invention relates to pharmaceutical formulations comprising fenofibrate. The invention also relates to pharmaceutical formulations comprising compositions of fenofibrate. Further the invention also relates to processes for preparing the compositions and/or formulations of fenofibrate and their methods of use, treatment and administration.

The present invention as discussed in various embodiments relates to stable and bioavailable formulations comprising fenofibrate.

In an aspect the invention provides pharmaceutical formulations comprising: (a) fenofibrate, at least one surfactant, and optionally at least one organic and/or inorganic substance; and (b) at least one carrier; said formulations further comprising one or more pharmaceutically acceptable excipients.

In an aspect the invention includes pharmaceutical formulations comprising fenofibrate and one or more pharmaceutical excipients, wherein said compositions comprise a hydrophilic material or polymer, and a water-soluble carrier.

In an aspect the invention includes pharmaceutical formulations comprising fenofibrate and one or more pharmaceutical excipients, wherein said compositions comprise a hydrophilic material or polymer, and a water-insoluble carrier.

In an aspect the invention includes pharmaceutical formulations comprising fenofibrate and one or more pharmaceutical excipients, wherein said compositions comprise a hydrophobic material or polymer, and a water-insoluble carrier.

In an aspect the invention includes pharmaceutical formulations comprising fenofibrate and one or more pharmaceutical excipients, wherein said formulations comprise a hydrophobic material or polymer, and a water-soluble carrier.

In an aspect the present invention provides pharmaceutical formulations comprising a spray dried emulsion of fenofibrate.

In an aspect the present invention provides pharmaceutical formulations of fenofibrate prepared by spray drying an emulsion comprising fenofibrate, at least one hydrophilic polymer and at least one surfactant onto inert substrate cores, or optionally collecting spray dried solid to obtain a pharmaceutical composition.

In an aspect the present invention provides pharmaceutical formulations comprising an emulsion of fenofibrate in one or more volatile organic solvents, which are immiscible with water, and water having at least one hydrophilic polymer dissolved in it, optionally having one or more of surfactants.

In an aspect the invention provides pharmaceutical formulations comprising fenofibrate, an organic substance such as stearic acid, a surfactant and one or more pharmaceutically acceptable excipients.

In an aspect the invention includes pharmaceutical formulations comprising fenofibrate, prepared by a process comprising the step of spray drying an emulsion comprising fenofibrate, or spraying an emulsion comprising fenofibrate onto a carrier and drying.

In aspects the invention provides processes for preparing pharmaceutical formulations of fenofibrate according to the present invention.

In an aspect the invention also provides methods of treating hypertriglyceridemia and/or hypercholesterolemia, using the pharmaceutical formulations of fenofibrate according to the present invention.

In an aspect the pharmaceutical formulations of fenofibrate according to the present invention additionally comprise an HMG-CoA reductase inhibitor, selected from the group comprising lovastatin, fluvastatin, rosuvastatin, pravastatin, cerivastatin, pitavastatin, atorvastatin, simvastatin, their pharmaceutically acceptable salts and derivatives thereof.

An aspect of the invention provides a pharmaceutical formulation comprising a dispersion containing fenofibrate and at least one surfactant, optionally combined with one or more solid organic or inorganic excipients.

An aspect of the invention provides a process for preparing a pharmaceutical formulation, comprising forming an emulsion containing fenofibrate, a surfactant, an aqueous fluid, and an organic fluid, combining the emulsion with a pharmaceutically acceptable solid, and removing volatile components to form a fenofibrate-containing solid.

An aspect of the invention provides a process for preparing a pharmaceutical formulation, comprising forming an dispersion containing fenofibrate and a liquid low-melting compound, solidifying the dispersion, and combining with one or more pharmaceutically acceptable excipients.

### DETAILED DESCRIPTION

The present invention relates to pharmaceutical formulations comprising fenofibrate. The invention also relates to pharmaceutical formulations comprising compositions of fenofibrate. Further the invention also relates to processes for preparing the compositions and/or formulations of fenofibrate and their methods of use, treatment and administration.

The present invention as discussed in various embodiments relates to stable and bioavailable formulations comprising fenofibrate.

“Fenofibrate” as employed herein refers to fenofibrate, its derivatives, prodrugs, active metabolites, and/or its polymorphs, solvates, hydrates, enantiomers, racemates and mixtures thereof. Further, it also includes amorphous or crystalline polymorphic forms of fenofibrate, and mixtures thereof.

In an aspect the invention provides pharmaceutical formulations comprising: (a) fenofibrate, at least one surfactant, and optionally along with at least one organic and/or inorganic substance; and (b) at least one carrier; said formulation further comprising one or more pharmaceutically acceptable excipients.

In an aspect the invention includes pharmaceutical formulations comprising fenofibrate and one or more pharmaceutical excipients, wherein said compositions comprise a hydrophilic material or polymer, and a water-soluble carrier.

In an aspect the invention includes pharmaceutical formulations comprising fenofibrate and one or more pharmaceutical excipients, wherein said compositions comprise a hydrophilic material or polymer, and a water-insoluble carrier.

In an aspect the invention includes pharmaceutical formulations comprising fenofibrate and one or more pharmaceutical excipients, wherein said compositions comprise a hydrophobic material or polymer, and a water-insoluble carrier.

In an aspect the invention includes pharmaceutical formulations comprising fenofibrate and one or more pharmaceutical excipients, wherein said formulations comprise a hydrophobic material or polymer, and a water-soluble carrier.

“Water-insoluble carrier” as used herein refers to excipients that do not substantially dissolve in water. Suitable examples include but are not limited to microcrystalline cellulose, silicified microcrystalline cellulose, dicalcium phosphate, partially pregelatinized starch, pregelatinized starch, colloidal silicon dioxide, and the like, their derivatives, and mixtures thereof.

“Water-soluble carrier” as used herein refers to excipients that substantially dissolve in water. Suitable examples include but are not limited to sucrose, dextrose, lactose, mannitol, sorbitol, and the like, their derivatives, and mixtures thereof.

One or more ‘inorganic substance’ as used herein refers to, but is not limited to, one or more of oxides, hydroxides, silicates, nitrides, carbonates, and the like, such as of metals.

Embodiments of ‘inorganic substance’ include but are not limited to one or more of aluminium oxide, magnesium oxide, silicon dioxide, colloidal silica, silicon dioxide fumed, calcium silicate, calcium carbonate, calcium phosphate, calcium sulfate, magnesium aluminum silicate, magnesium aluminium trisilicate, sodium carbonate, iron oxide, ferric hydroxide, their derivatives, and mixtures thereof.

One or more ‘organic substance’ as used herein refers to, but is not limited to, stearic acid, polyoxylglycerides, lauroyl macrogolglycerides, stearoyl macrogolglycerides, triglycerides, mixture of mono-, di- and tri-glycerides, oils, fatty acids, paraffins, hydrogenated vegetable oils, polyoxyethylene derivatives of organic ethers, and the like, their derivatives and mixtures thereof. An example of lauroyl macrogolglycerides includes the commercially available Gelucire® 44/14. An example of stearoyl macrogolglycerides includes the commercially available Gelucire® 50/13. Gelucire products are sold by Gattefossé, St-Priest France.

‘Surfactant’ as used herein includes ionic and nonionic types. Ionic surfactants may be anionic, cationic, or zwitterionic. Anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids. Non-limiting examples include chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium lauryl sulfate (SLS), also called sodium dodecyl sulfate (SDS).

Examples of amphoteric and zwitterionic surfactants include but are not limited to carboxy, sulfonate, sulfate, phosphate, and phosphonate compounds. Examples are alkylimino acetates and iminodialkanoates and aminoalkanoates, imidazolinium and ammonium derivatives, betaines, sultaines, hydroxysultaines, alkyl sarcosinates and alkanoyl sarcosinates, and the like.

Nonionic surfactants include polyoxyethylene castor oil derivatives, polyoxyethylene sorbitan fatty acid esters (e.g., the commercially available Tween® products, including Tween 20 and Tween 800, from ICI Speciality Chemicals), sorbitan esters (e.g., sorbitan fatty acid esters such as the commercially available Span® products from ICI Americas, including Span 20, 60, 80, and 85), poloxamers (e.g., Pluronic® products F68, F127 and F108Q, which are block copolymers of ethylene oxide and propylene oxide, from BASF Corporation), poloxamines (e.g., Tetronic® 908, also known as poloxamine 908, which is a tetrafunctional block copolymer derived from sequential addition of propylene oxide and ethylene oxide to ethylenediamine from BASF Wyandotte Corporation, Parsippany, N.J. USA), and Tetronic™ 15080 (T-1508) from BASF Wyandotte Corporation.

Examples of useful cationic surfactants include, but are not limited to, polymers, biopolymers, polysaccharides, cellulosics, alginates, phospholipids, and nonpolymeric compounds, such as zwitterionic stabilizers, poly-n-methylpyridinium, anthrylpyridinium chloride, cationic phospholipids, chitosan, polylysine, polyvinylimidazole, polybrene, polymethylmethacrylate, polyvinylpyrrolidone-2-dimethylaminoethyl methacrylate, lysozyme, long-chain polymers such as alginic acid, carrageenan (FMC Corp.), and POLYOX™ (Dow Chemical Co., Midland, Mich. USA), cationic lipids, sulfonium, phosphonium, and quarternary ammonium compounds, such as stearyltrimethylammonium chloride, and benzyl-di-(2-chloroethyl)ethylammonium bromide.

In an aspect pharmaceutical formulations of the present invention comprise at least two surfactants.

In an aspect the invention relates to pharmaceutical formulations comprising fenofibrate, wherein an embodiment comprises compositions of fenofibrate with stearic acid, further comprising a surfactant optionally along with a carrier and other pharmaceutically acceptable excipients.

In an aspect the invention relates to pharmaceutical formulations comprising fenofibrate, wherein an embodiment comprises dispersions of fenofibrate in stearic acid, further comprising a surfactant, a Sorensen buffer (mixture of 0.2 M Na₂HPO₄ and 0.2 M NaH₂PO₄ to achieve a desired pH value between 5.8 and 8), and optionally other pharmaceutically acceptable excipients. The presence of a Sorensen buffer promotes in situ formation of sodium stearate, which results in a better solubility. Variations of the buffers can use other metal salts, such as potassium salts, for either component.

Embodiments of the invention also relates to pharmaceutical compositions of fenofibrate, comprising fenofibrate, which is melt-granulated with an one or more organic and/or inorganic substance comprising a surfactant within or outside the melt granulate, wherein the granules are blended with a carrier and one or more other pharmaceutically acceptable excipients.

‘Melt granulate’ as used herein refers to a composition comprising fenofibrate, one or more organic and/or inorganic substance which has a low melting point, and a surfactant.

In aspects the invention includes processes for preparing pharmaceutical formulations of fenofibrate according to the present invention.

In an aspect a process for preparing pharmaceutical formulations comprises: (a) forming a dispersion of fenofibrate, one or more organic or inorganic substance along with a surfactant in water or organic solvent or mixtures thereof; (b) spraying the dispersion onto a carrier and drying; and (c) combining the dried material of step (b) and with one or more pharmaceutically acceptable excipients.

In an aspect a process for preparing pharmaceutical formulations comprises: (a) forming a dispersion of fenofibrate, and one or more organic or inorganic substances along with a surfactant and a carrier in water or organic solvent or mixtures thereof; (b) spray drying the dispersion; and (c) combining the dried material of step (b) along with one or more pharmaceutically acceptable excipients.

In an aspect a process for preparing pharmaceutical formulations comprises: (a) forming a solution of fenofibrate, and one or more organic or inorganic substances along with a surfactant in water or organic solvent or mixtures thereof; (b) spraying the solution onto a carrier to obtain a co-precipitate, and drying; and (c) combining the dried material of step (b) along with one or more pharmaceutically acceptable excipients.

In an aspect compositions comprising fenofibrate in the form of granules, powder, pellets, spheres, and the like are compressed into tablets or filled into capsules.

Being drug with limited solubility, particle sizes of fenofibrate would impact its solubility. The smaller the particle size, the greater the surface area resulting in higher solubility and bioavailability.

As used herein, particle size is determined on the basis of the weight or volume average particle sizes as measured by particle size measuring techniques well known to those skilled in the art. Such techniques include, for example, sedimentation field flow fractionation, photon correlation spectroscopy, laser light scattering such as using a Malvern particle size analyzer (Malvern Instruments Ltd., Malvern, Worcestershire, United Kingdom), and disk centrifugation.

In an embodiment, the present invention relates to pharmaceutical formulations comprising fenofibrate particles having effective average particle sizes greater than about 100 nm, or about 500 nm, or about 800 nm. “Effective average particle size” as used herein refers to at least 50% by weight of the drug particles having particle sizes greater than the specified size.

In an aspect the pharmaceutical formulations of the present invention comprise spray dried emulsions of fenofibrate.

In an aspect a pharmaceutical formulation of fenofibrate is prepared by spray drying an emulsion comprising fenofibrate, at least one hydrophilic polymer, and at least one surfactant, onto inert substrate cores, or optionally collecting the spray dried solid to obtain a pharmaceutical composition.

In an aspect a pharmaceutical formulation comprises an emulsion of fenofibrate in one or more of volatile organic solvents which are immiscible with water, and water having at least one hydrophilic polymer dissolved in it, optionally having one or more of surfactants.

In an aspect a process for preparing pharmaceutical formulations comprises the steps of: (a) dissolving fenofibrate in a volatile organic solvent optionally having one or more surfactant (the dispersed phase); (b) dissolving one or more hydrophilic polymers in water, optionally having one or more surfactant (the continuous phase); (c) combining the materials of step b) and step a) under continuous mixing to get an emulsion; (d) spray drying the emulsion to obtain a powder; and (e) combining the dried material of step (d) along with one or more pharmaceutically acceptable excipient(s).

In aspects the invention includes processes for preparing pharmaceutical formulations of fenofibrate, prepared by spraying an emulsion comprising fenofibrate, at least one hydrophilic polymer, and at least one surfactant, onto inert substrate cores, or optionally collecting a spray dried solid to obtain a pharmaceutical composition.

‘Emulsion’ as used herein refers to mixtures of two immiscible and/or unblendable liquids. One liquid, i.e., the dispersed phase, is dispersed in the other, i.e., the continuous phase.

In an embodiment, an emulsion of the present invention comprises fenofibrate dissolved in a solvent which is immiscible with water, and water having at least one hydrophilic polymer dissolved therein.

Embodiments of processes for preparing pharmaceutical emulsions of fenofibrate include one or more of the following steps:

a) dissolving fenofibrate in a volatile organic solvent (i.e., the dispersed phase), optionally together with one or more surfactants;

b) dissolving one or more hydrophilic polymers in water, optionally together with one or more surfactants (i.e., the continuous phase);

c) combining step b) and step a) under continuous mixing to produce an emulsion; and

d) spray drying the emulsion.

The emulsion can be spray dried onto an inert substrate core and converted into suitable dosage forms or the solid can be collected after drying and converted into suitable dosage forms by any method known to the skilled artisan. For example, the emulsion can be sprayed on an inert substrate core to form granulate. The granulate can further be compressed into tablets and/or can be filled into capsules. Alternatively the spray-dried solid, optionally with any desired pharmaceutical excipients, can be compressed into tablets and/or can be filled into capsules.

‘Inert substrate cores’ as used herein refers to any pharmacologically inert solid. Examples of various pharmaceutically acceptable excipients that can comprise the core include but are not limited to sucrose, dextrose, lactose, anhydrous lactose, spray-dried lactose, lactose monohydrate, mannitol, starches, sorbitol, polyvinylchloride, polystyrene, glass, silicon dioxide, calcium phosphate dihydrate, dicalcium phosphate, calcium sulfate dihydrate, microcrystalline cellulose, cellulose derivatives, calcium carbonate, dibasic calcium phosphate anhydrous, dibasic calcium phosphate monohydrate, tribasic calcium phosphate, magnesium carbonate, magnesium oxide, and the like, their derivatives and mixtures thereof.

In embodiments, the cores may be optionally seal coated to increase the strength of the core to withstand the mechanical pressures during processing.

In embodiments, inert cores can be any inert particles commercially available in form of pellets, beads, granules, minitablets, spheres, spheroids and/or modifications thereof, and can be used as a starting material.

The fenofibrate formulations may be prepared using techniques known to those skilled in the art, including, but not limited to, the techniques of extrusion and spheronization, wet granulation, fluid bed granulation, and rotary bed granulation. In addition, the core may also be prepared by building the fenofibrate composition (drug plus excipients) on an inert core using a drug-layering technique such as powder coating, or applying the fenofibrate composition by spraying a solution or dispersion of fenofibrate in an appropriate binder solution onto inert cores in a fluidized bed such as with a Wurster coater or a rotary processor.

Non-limiting examples of a “solvent” and “volatile organic solvent” as used herein include methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, cyclohexane, pentane, hexane, heptane, carbon tetrachloride, p-xylene, toluene, benzene, ether, methyl t-butyl ether (MTBE), chloroform, dimethylsulphoxide, dimethylformamide, tetrahydrofuran, and mixtures thereof.

Non-limiting examples of “hydrophilic polymers” as used herein include sodium carboxymethylcelluloses, hydroxypropyl methylcelluloses, hydroxyethylcelluloses, hydroxypropylcelluloses, carboxymethylamide, potassium methacrylate-divinylbenzene copolymer, polymethylmethacrylates, polyvinylpyrrolidones, polyvinylalcohols, methylcelluloses, carboxymethylcelluloses, polyoxyethyleneglycols, xanthan gum, carbomers, Polyox, hydrocolloids such as natural or synthetic gums, cellulose derivatives other than those listed above, carbohydrate-based substances such as acacia, gum tragacanth, locust bean gum, guar gum, agar, pectin, carrageen, soluble alginates, carboxypolymethylene, and the like, and mixtures thereof.

In general embodiments of the present invention, the formulation as a whole, or at least partially, comprises formulations, which are monolithic and/or multiparticulate, in matrix and/or reservoir form, or a combination thereof.

In an aspect pharmaceutical formulations of fenofibrate are in the form of tablets or capsules, or multiparticulates filled into capsules.

In an aspect pharmaceutical formulations of fenofibrate as multiparticulates are in the form of granules, spheroids, pellets, powder, minitablets and mixtures thereof.

In general embodiments of the present invention, formulations comprise fenofibrate and one or more pharmaceutically acceptable excipients such as binders, diluents, lubricant/glidants, disintegrating agents, surfactants, solvents, and coloring agents.

Non-limiting examples of “binders” include one or more of gum acacia, cholesterol, tragacanth, stearic acid, gelatin, casein, lecithin (phosphatides), carboxymethylcellulose calcium, carboxymethylcellulose sodium, methylcelluloses, hydroxyethylcelluloses, hydroxypropylcelluloses, hydroxypropyl methycellulose phthalates, microcrystalline celluloses, noncrystalline celluloses, polyvinylpyrrolidones (povidones or PVP), cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, polyvinyl alcohols, and mixtures thereof.

Non-limiting examples of “diluents” as used herein include calcium carbonate, calcium phosphate dibasic, calcium phosphate tribasic, calcium sulfate, microcrystalline cellulose, powdered cellulose, dextrates, dextrins, dextrose excipients, fructose, kaolin, lactitol, lactose, mannitol, sorbitol, starches, sucrose, and mixtures thereof.

Non-limiting examples of lubricants/glidants include colloidal silicon dioxide, stearic acid, magnesium stearate, calcium stearate, talc, hydrogenated castor oil, and mixtures thereof.

Non-limiting examples of disintegrants comprise starches or modified starches such as starch, modified starch, croscarmellose sodium, crospovidone and sodium starch glycolate.

Coloring agents include FDA approved colorants and examples are iron oxides, lake of tartrazine, allura red, lake of quinoline yellow, and lake of erythrosine.

In yet another aspect the pharmaceutical formulations of fenofibrate according to the present invention additionally comprise an HMG-CoA reductase inhibitor, selected from the group comprising lovastatin, fluvastatin, rosuvastatin, pravastatin, cerivastatin, pitavastatin, atorvastatin, simvastatin, their pharmaceutically acceptable salts and derivatives thereof.

In yet another general aspect, the present invention relates to methods of using pharmaceutical formulations of the present invention, such as for the treatment of hypertriglyceridemia and hypercholesterolemia.

Embodiments of methods of using pharmaceutical formulations of the present invention may include use in combination with a HMG-CoA reductase inhibitor for the treatment of hypertriglyceridemia and hypercholesterolemia.

The formulations can be packaged using appropriate packaging materials such as containers, including lids, composed of polyethylene (high density polyethylene or low density polyethylene), polypropylene, glass, stainless steel bottles, etc. Also useful are various blisters or strips composed of aluminium or high-density polypropylene, or polyvinylchloride, or polyvinylchloride (PVC) coated with polyvinylidene dichloride (PVDC), generally termed PVC/PVDC. Different grades of PVC/PVDC are available as PVC/PVDC 40 gsm, PVC/PVDC 60 gsm, PVC/PVDC 90 gsm, etc., where “gsm” indicates the grams of PVDC coating per square meter of PVC film.

Drug release characteristics of pharmaceutical products can be determined, such as by using Test 711 “Dissolution,” in United States Pharmacopeia 29, United States Pharmacopeial Convention, Inc., Rockville, Md., pages 2673-2682, 2005 (“USP”).

The following examples illustrate certain specific aspects and embodiments of the invention and demonstrate the practice and advantages thereof. It is to be understood that the examples are given by way of illustration only and are not intended to limit the scope of the invention in any manner.

### EXAMPLE 1

### Fenofibrate 145 mg Tablets

| | |
|---|---|
| Ingredient | mg/Tablet |

Dispersion

| | |
|---|---|
| Fenofibrate | 145 |
| Sodium lauryl sulphate (SLS) | 25 |
| Magnesium aluminium trisilicate | 40 |
| Sorbitan monolaurate (Span ™ 20) | 10 |
| Water‡ | q.s. |
| Methylene chloride‡ | q.s. |

Granulation

| | |
|---|---|
| Partially pregelatinised starch (PPG) | 325 |
| Colloidal silicon dioxide (Aerosil ™ 200) | 10 |

Extragranular

| | |
|---|---|
| Microcrystalline cellulose (MCC PH 102) | 105 |
| Crospovidone XL 10 | 150 |
| Sodium lauryl sulphate (SLS) | 20 |
| Silicified microcrystalline cellulose | 20 |
| (Prosolv HD90)$ | |
| Colloidal silicon dioxide (Aerosil ™ 200) | 6.5 |
| Magnesium stearate | 3.5 |
| Opadry ™ AMB* | 26 |

*Opadry AMB contains polyvinyl alcohol, lecithin, xanthan gum, talc, and is supplied by Colorcon.
$Prosolv HD 90 is silicified microcrystalline cellulose and is supplied by PenWest Co.
‡Evaporates during processing.

Manufacturing Process:

1. Preparation of drug dispersion:
1.1. Magnesium aluminium silicate was dispersed in water.
1.2. SLS was added to step 1.1
1.3. Span 20 was added to step 1.2 and kept under continuous stirring.
1.4. Fenofibrate was dissolved in methylene chloride, added to the step 1.3 dispersion, and homogenized for 2 hours. (Particle size by Malvern: D₅₀=0.994 μm)

2. Granulation:
2.1. Partially pregelatinised starch and Aerosil were sifted through an ASTM #40 mesh sieve and loaded into a fluidized bed processor
2.2. The step 2.1 materials were granulated by adding the dispersion prepared in step 1.4
2.3. The granules obtained were dried and sifted through an ASTM #40 mesh sieve.

3. Tablet formation:
3.1. Extragranular MCC PH102, Prosolve, crospovidone, SLS and Aerosil were passed through an ASTM #40 mesh sieve and blended with the granules obtained in step 2.3 for 10 minutes.
3.2. Magnesium stearate was passed through an ASTM #60 mesh sieve and blended with the materials of step 3.1 for 5 minutes.
3.3. The lubricated blend of step 3.2 was compressed into tablets.
3.4. The tablets of step 3.3 were coated with an aqueous dispersion of Opadry AMB until a 3% weight gain was obtained.

### EXAMPLE 2

### Fenofibrate 145 mg Tablets

| | |
|---|---|
| Ingredient | mg/Tablet |

Dispersion

| | |
|---|---|
| Fenofibrate | 145 |
| Sodium lauryl sulphate (SLS) | 25 |
| Magnesium aluminium silicate | 40 |
| Sorbitan monolaurate (Span 20) | 20 |
| Water‡ | q.s |
| Methylene chloride‡ | q.s |

Granulation

| | |
|---|---|
| Partially pregelatinised starch (PPG) | 325 |
| Colloidal silicon dioxide (Aerosil ™ 200) | 10 |

Extragranular

| | |
|---|---|
| Microcrystalline cellulose (MCC PH 102) | 95 |
| Crospovidone XL 10 | 150 |
| Sodium lauryl sulphate (SLS) | 20 |
| Silicified microcrystalline cellulose | 20 |
| (Prosolv HD90) | |
| Colloidal silicon dioxide (Aerosil ™ 200) | 6.5 |
| Magnesium stearate | 3.5 |
| Opadry AMB | 26 |

‡Evaporates during processing.

Manufacturing process: similar to that for Example 1.

The tablets of Examples 1 and 2, and TriCor® 145 mg tablets were subjected to dissolution testing using the USP procedure in 1000 ml of 25 mM sodium lauryl sulphate (SLS) in water, 50 RPM stirring, in USP II dissolution apparatus. The cumulative percentages of drug dissolved are shown in Table 1.

TABLE 1

| | Cumulative Percent of Drug Dissolved | | |
|---|---|---|---|
| Minutes | TriCor ® 145 mg | Example 1 | Example 2 |
| 10 | 40 | 77 | 57 |
| 20 | 82 | 90 | 79 |
| 30 | 92 | 93 | 84 |
| 45 | 94 | 94 | 86 |

### EXAMPLE 3

### Fenofibrate 145 mg Tablets

| | |
|---|---|
| Ingredient | mg/Tablet |

Dispersion

| | |
|---|---|
| Fenofibrate | 145 |
| Stearic acid | 36.25 |
| Sodium lauryl sulphate (SLS) | 200 |
| Disodium hydrogen phosphate | 37.78 |
| (Na₂HPO₄) | |
| Potassium dihydrogen phosphate | 36.32 |
| (KH₂PO₄) | |
| Water‡ | q.s. |

Granulation

| | |
|---|---|
| Partially pregelatinised starch (PPG) | 275 |
| Colloidal silicon dioxide (Aerosil ™ 200) | 10 |

Extragranular

| | |
|---|---|
| Microcrystalline cellulose (MCC) PH 102 | 35 |
| Crospovidone XL 10 | 150 |
| Colloidal silicon dioxide (Aerosil ™ 200) | 6.00 |
| Magnesium stearate | 3.50 |

‡Evaporates during processing.

Manufacturing Process:

1. Dispersion:
1.1. Stearic acid was melted by heating.
1.2. Fenofibrate was added to step 1.1 and heating was continued until a homogeneous dispersion was formed.
1.3. Solution of step 1.2 was cooled to room temperature and the solid was milled and sifted through an ASTM #40 mesh sieve.
1.4. Disodium hydrogen phosphate and potassium dihydrogen phosphate were dissolved in water to form a solution having pH 6.8.
1.5 Sodium lauryl sulphate and step 1.3 solid were added to step 1.4 buffer and homogenized for 2 hours, then the dispersion was subjected to bead milling.

2. Granulation:
2.1. Partially pregelatinised starch and Aerosil were sifted through an ASTM #40 mesh sieve and loaded into a fluidized bed processor.
2.2 The materials in 2.1 were granulated using the dispersion prepared in step 1.5.
2.3. The granules obtained were dried and sifted through an ASTM #40 mesh sieve.

3. Tablet formation:
3.1. Extragranular MCC PH102, crospovidone, and Aerosil were passed through an ASTM #40 mesh sieve and blended with the granules obtained in 2.3 for 10 minutes.
3.2. Magnesium stearate was passed through an ASTM #60 mesh sieve and blended with the materials of 3.1 for 5 minutes.
3.3. The lubricated blend of 3.2 was compressed into tablets.

### EXAMPLE 4

### Fenofibrate 145 mg Tablets

mg/Tablet

| Ingredient | 4A | 4B |
|---|---|---|
| Fenofibrate | 145 | 145 |
| Silicon dioxide | 72.5 | — |
| Calcium silicate | — | 72.5 |
| Sodium lauryl sulphate (SLS) | 20 | 20 |
| Water‡ | q.s. | q.s. |
| Methylene chloride‡ | q.s. | q.s. |

Homogenization

| | | |
|---|---|---|
| SLS | 20 | 20 |

Granulation

| | | |
|---|---|---|
| Pregelatinised starch | 250 | 250 |

Extragranular materials

| | | |
|---|---|---|
| Microcrystalline cellulose | 35 | 35 |
| Crospovidone | 150 | 150 |
| Colloidal silicon dioxide (Aerosil ™ 200) | 6 | 6 |
| Magnesium stearate | 3.5 | 3.5 |

Film Coating

| | | |
|---|---|---|
| Opadry ™ AMB | 21 | 21 |

‡Evaporates during processing.

Manufacturing Process:

1. Preparation of drug dispersion:
1.1. Fenofibrate was dissolved in dichloromethane.
1.2. SLS was dissolved in water.
1.3. Silicon dioxide (for 4A) or calcium silicate (for 4B) was dispersed in solution 1.2.
1.4. The materials prepared in 1.1 and 1.3 were mixed together, during which precipitation occurred.

2. Homogenization:
2.1. The precipitate of 1.4 was homogenized with sodium lauryl sulphate.

3. Granulation:
3.1. Pregelatinised starch was sifted through an ASTM #40 mesh sieve and loaded into a fluid bed processor.
3.2. The material of 3.1 was granulated with 2.1 dispersion and the granules were dried and sifted through an ASTM #40 mesh sieve.

4. Tablet formation:
4.1. Extragranular MCC, crospovidone and Aerosil were sifted through an ASTM #40 mesh sieve and blended with the granules of step 3.2 for 10 minutes.
4.2. Magnesium stearate was sifted through an ASTM #60 mesh sieve and blended with 4.1 materials.
4.3. The lubricated blend of 4.2 was compressed into tablets and coated with Opadry™ AMB in water.

### EXAMPLE 5

### Emulsion Composition

| Ingredient | Wt. Percent |
|---|---|
| Fenofibrate | 3.33 |
| Hydroxyethyl cellulose L250 | 1.17 |
| Sodium carboxymethylcellulose 7LF | 1.17 |
| Polysorbate 80 (Tween ™ 80) | 0.92 |
| Sodium lauryl sulfate | 0.92 |
| Dichloromethane* | 27.75 |
| Isopropyl alcohol* | 3.69 |
| Water* | 61.06 |

*Evaporates during processing.

Manufacturing Process:

1. Hydroxyethylcellulose L250, sodium carboxymethylcellulose 7LF and sodium lauryl sulfate were added to water and stirred continuously to get a clear solution, and then isopropyl alcohol was added.

2. Polysorbate 80 and fenofibrate were dissolved in dichloromethane.

3. Step 2 was added slowly to step 1 under homogenization conditions to produce a clear emulsion.

4. Step 3 was subjected to spray drying to get a spray-dried emulsion mixture comprising fenofibrate.

### EXAMPLE 6

### Fenofibrate 145 mg Tablets

| Ingredient | Wt. Percent |
|---|---|
| Spray-dried emulsion mixture of | 40.88 |
| fenofibrate (Example 5) | |
| Colloidal silicon dioxide | 1.25 |
| Microcrystalline cellulose | 25 |
| (Avicel ™ PH102) | |
| Silicified microcrystalline | 25 |
| cellulose (Prosolv HD90) | |
| Croscarmellose sodium | 7 |
| Magnesium stearate | 0.88 |

Manufacturing Process:

1. Co-sifted colloidal silicon dioxide, microcrystalline cellulose, spray dried composition of fenofibrate of Example 5, silicified microcrystalline cellulose and croscarmellose sodium through a #30 mesh sieve and mixed for 10 minutes in a blender.

2. Magnesium stearate was added to step 1 and mixed for 5 minutes.

3. The step 2 blend was compressed into tablets using a rotary compression machine.

### EXAMPLE 7

### Fenofibrate Emulsion Coating on Inert Tablet Core

| Ingredient | Wt. Percent |
|---|---|
| Colloidal silicon dioxide | 1.49 |
| Microcrystalline cellulose | 44.78 |
| (Avicel PH102)* | |
| Silicified microcrystalline | 44.78 |
| cellulose (Prosolv HD90) | |
| Croscarmellose sodium | 7.91 |
| Magnesium stearate | 1.04 |

*Avicel PH 102 is supplied by FMC Biopolymers.

Manufacturing Procedure:

1. Co-sifted colloidal silicon dioxide, microcrystalline cellulose, silicified microcrystalline cellulose and croscarmellose sodium through a #30 mesh sieve and mixed for 10 minutes in a blender.

2. Magnesium stearate was added to step 1 and mixed for 5 minutes.

3. The step 2 blend was compressed into tablet cores using a rotary compression machine.

4. Emulsion prepared in Example 5 was sprayed onto the inert core tablets of step 3 to provide a drug layer.

### EXAMPLE 8

### Fenofibrate Emulsion Coatings on Inner Core, with Outer Tablet Coating

Emulsions:

Wt. Percent

| Ingredient | 8A | 8B |
|---|---|---|
| Fenofibrate | 2.22 | 3.33 |
| Hydroxyethyl cellulose L250 | 0.78 | 1.17 |
| Sodium carboxy | 0.78 | 1.17 |
| methylcellulose 7LF | | |
| Polysorbate 80 (Tween 80) | 0.61 | 0.92 |
| Sodium lauryl sulfate | 0.61 | 0.92 |
| Dichloromethane | 28.5 | 27.75 |
| Isopropyl alcohol | 3.8 | 3.7 |
| Water | 62.7 | 61.05 |

Manufacturing Process:

1. Hydroxyethylcellulose L250, sodium carboxymethylcellulose 7LF and sodium lauryl sulfate were added to water and stirred continuously to get a clear solution, and then isopropyl alcohol was added.

2. Polysorbate 80 and fenofibrate were dissolved in dichloromethane.

3. Added slowly step 2 to step 1 under homogenization conditions to produce a clear emulsion.

Core Tablets:

| Ingredient | Wt. Percent |
|---|---|
| Colloidal silicon dioxide | 1.19 |
| Microcrystalline cellulose | 76.19 |
| (Avicel PH101) | |
| Croscarmellose sodium | 2.86 |
| Emulsion 8A solid* | 12.98 |
| Croscarmellose sodium | 5.95 |
| Magnesium stearate | 0.83 |

*Solid remaining after evaporating solvents.

Manufacturing Process:

1. Colloidal silicon dioxide, microcrystalline cellulose, and croscarmellose sodium were co-sifted through a #30 mesh sieve and mixed in a blender for 10 minutes.

2. The step 1 blend was wet granulated by spraying Emulsion A onto it.

3. The step 2 wet granulate was subsequently dried and sized using a #20 mesh sieve.

4. Croscarmellose sodium and magnesium stearate were co-sifted through a #40 mesh sieve.

5. The dried granules of the step 3 were mixed with step 4 powders in a blender for 3 minutes.

6. The mixture of step 5 was compressed into tablets using a rotary compression machine.

Outer Drug Coating:

| Ingredient | Weight (mg) |
|---|---|
| Core tablet | 840 |
| Emulsion 8B | 218 |
| Total tablet | 1058 |

Manufacturing Process:

Compressed tablets of step 6 were coated with Emulsion 8B in tablet coating equipment until complete layering of the drug was achieved.

The final tablets had 48.33 mg of fenofibrate in the core and 96.67 mg of fenofibrate in the coating.

### EXAMPLE 9

### Fenofibrate Emulsion Coating on Multiparticulate Inner Core and as Outer Tablet Coat for Fenofibrate 145 mg Tablet

Emulsions:

Wt. Percent

| Ingredient | 9A | 9B |
|---|---|---|
| Fenofibrate | 2.22 | 3.33 |
| Hydroxyethyl cellulose | 0.78 | 1.17 |
| L250 | | |
| Sodium carboxy | 0.78 | 1.17 |
| methylcellulose 7LF | | |
| Polysorbate 80 (Tween | 0.61 | 0.92 |
| 80) | | |
| Sodium lauryl sulfate | 0.61 | 0.92 |
| Dichloromethane | 28.5 | 27.75 |
| Isopropyl alcohol | 3.8 | 3.7 |
| Water | 62.7 | 61.05 |

Manufacturing Process:

1. Hydroxyethylcellulose L250, sodium carboxymethylcellulose 7LF and sodium lauryl sulfate were added to water and stirred continuously to get a clear solution, and then isopropyl alcohol was added.

2. Polysorbate 80 and fenofibrate were dissolved in dichloromethane.

3. Added slowly step 2 to step 1 under homogenization conditions to produce a clear emulsion.

Core Tablets:

| Ingredient | Wt. Percent |
|---|---|
| Celphere CP 102 (microcrystalline | 35.29 |
| cellulose spheres)** | |
| Emulsion 9A solid* | 12.82 |

Tablet Additives

| | |
|---|---|
| Colloidal silicon dioxide | 0.47 |
| Silicified microcrystalline cellulose | 47.06 |
| (Prosolv HD90) | |
| Croscarmellose sodium | 3.53 |
| Magnesium stearate | 0.82 |

*Solid after evaporating solvents from the emulsion.
**Celphere CP 102 is supplied by Asahi Kasei Company.

Manufacturing Process:

1. Celphere CP 102 was placed in a fluidized bed coater (FBC) and fluidized using appropriate air pressure.

2. Emulsion A was sprayed into the FBC using a bottom spray assembly.

3. Colloidal silicon dioxide, silicified microcrystalline cellulose, and croscarmellose sodium were co-sifted through a #30 mesh sieve and mixed in a blender for 10 minutes.

4. After drying, step 2 drug loaded particles were mixed with the step 3 blend for 10 minutes.

5. Magnesium stearate was added to the step 4 mixture and mixed in a blender for 3 minutes.

6. The step 5 blend was compressed into core tablets comprising fenofibrate.

Outer Drug Coating:

| Ingredient | Weight (mg) |
|---|---|
| Core tablet | 840 |
| Emulsion 9B | 218 |
| Total weight | 1058 |

Manufacturing Process:

Compressed tablets of step 6 above were coated with Emulsion B in tablet coating equipment.

The final tablets had 48.33 mg of fenofibrate in the core and 96.67 mg of fenofibrate in the coating.

The coated tablets of Example 9 and TriCor® 145 mg tablets were subjected to dissolution testing using the USP procedure in 1000 ml of 25 mM sodium lauryl sulphate (SLS) in water, 50 RPM stirring, in USP II dissolution apparatus. The cumulative percentages of drug dissolved are shown in Table 2.

TABLE 2

Cumulative Percent of
Drug Dissolved

| Minutes | TriCor ® 145 mg | Example 9 |
|---|---|---|
| 5 | 24 | 28 |
| 10 | 58 | 55 |
| 20 | 85 | 82 |
| 30 | 89 | 90 |
| 45 | 93 | 96 |

We claim:

1. A pharmaceutical formulation comprising a dispersion containing fenofibrate and at least one surfactant, optionally combined with one or more solid organic or inorganic excipients.

2. The pharmaceutical formulation of claim 1, wherein a dispersion further contains a hydrophilic polymer.

3. The pharmaceutical formulation according to claim 1, wherein a dispersion is an emulsion.

4. The pharmaceutical formulation of claim 1, wherein a solid organic or inorganic excipient is granulated with a dispersion in fluid form.

5. The pharmaceutical formulation of claim 1, wherein a dispersion in fluid form is coated onto a solid organic or inorganic excipient.

6. The pharmaceutical formulation of claim 1, comprising fenofibrate and a surfactant dispersed in a low-melting solid.

7. The pharmaceutical formulation of claim 1, comprising fenofibrate dispersed in a low-melting solid, the solid being combined with a surfactant.

8. The pharmaceutical formulation of claim 1, comprising a solvent-free emulsion containing fenofibrate and a surfactant, and one or more solid organic or inorganic excipients.

9. The pharmaceutical formulation of claim 1, comprising a solid dispersion of fenofibrate and a surfactant in a low-melting solid, and one or more solid organic or inorganic excipients.

10. The pharmaceutical formulation of claim 1, comprising a solid dispersion of fenofibrate in a low-melting solid, combined with a surfactant and one or more solid organic or inorganic excipients.

11. The pharmaceutical formulation of claim 1, further comprising an HMG-Coa reductase inhibitor.

12. A process for preparing a pharmaceutical formulation, comprising forming an emulsion containing fenofibrate, a surfactant, an aqueous fluid, and an organic fluid, combining the emulsion with a pharmaceutically acceptable solid, and removing volatile components to form a fenofibrate-containing solid.

13. The process of claim 12, wherein the emulsion is coated onto solid particles.

14. The process of claim 12, wherein the emulsion is used to granulate a powder.

15. The process of claim 12, wherein a fenofibrate-containing solid is combined with one or more pharmaceutically acceptable excipients and compressed into tablets or filled into capsules.

16. The process of claim 15, further comprising combining a fenofibrate-containing solid with an HMG-Coa reductase inhibitor.

17. The process of claim 12, wherein a fenofibrate-containing solid is combined with one or more pharmaceutically acceptable excipients and additional fenofibrate, and compressed into tablets or filled into capsules.

18. A process for preparing a pharmaceutical formulation, comprising forming an dispersion containing fenofibrate and a liquid low-melting compound, solidifying the dispersion, and combining with one or more pharmaceutically acceptable excipients.

19. The process of claim 18, wherein a dispersion further contains a surfactant.

20. The process of claim 18, wherein a pharmaceutically acceptable excipient comprises a surfactant.
